(19) United States
(12) Patent Application Publication
(10) Pub. No.: US 2008/0161824 A1
(43) Pub. Date: Jul. 3, 2008

(54) System and method for performing femoral sizing through navigation

(75) Inventor: Troy Allen McMillen, Milford, PA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/645,940

(22) Filed: Dec. 27, 2006

(51) Int. Cl. A61B 17/58; A61B 17/00

(52) U.S. Cl. 606/102; 606/87; 606/88

(57) ABSTRACT

An apparatus, method, and system for sizing a distal portion of a patient's femur during knee arthroplasty. The femur is sized by positioning the patient such that the distal femur portion of the patient is in a field of view of a sensor array; attaching a femoral sizer to the distal femur portion, the femoral sizer including a tracker that is operable to provide signals to the sensor array; manipulating the sizer while observing a display that displays an image based on the signals provided by the tracker to the sensor array; and determining the size of the distal portion of the femur by observing the sizer in response to an indication provided on the display.

### FIELD OF THE INVENTION

The present invention relates to methods and tools used in knee arthroplasty. More particularly, the invention relates to methods and tools used in knee surgery involving the installation of an artificial femoral component.

### BACKGROUND OF THE INVENTION

Total knee arthroplasty involves the replacement of portions of the patellar, femur and tibia with artificial components. In particular, a proximal portion of the tibia and a distal portion of the femur are cut away (resected) and replaced with artificial components.

As used herein, when referring to bones or other body parts, the term “proximal” means closest to the heart and the term “distal” means more distant from the heart. When referring to tools and instruments, the term “proximal” means closest to the practitioner and the term “distal” means distant from the practitioner. However, when a tool or instrument is fixated to a bone or other body part the terms “proximal” and “distal” are applied to the tool or instrument as if the tool or instrument were itself a bone or body part.

There are several types of knee prostheses known in the art. One type is sometimes referred to as a “resurfacing type.” In these prostheses, the articular surface of the distal femur and proximal tibia are “resurfaced” with respective metal and plastic condylar-type articular bearing components.

The femoral component is typically a metallic alloy construction (e.g. cobalt-chrome alloy or 6A14V titanium alloy) and provides medial and lateral condylar bearing surfaces of multi-radii design of similar shape and geometry as the natural distal femur or femoral-side of the knee joint.

One important aspect of knee arthroplasty procedures is the correct resection of the distal femur and proximal tibia. These resections must provide planes which are correctly oriented in order to properly accept the prosthetic components. Among the factors that are considered when assessing resection of the distal femur and proximal tibia are the proximal-distal location of the resection planes, the varus-valgus angle of the planes, and the change in relative orientation of the planes in response to change in flexion-extension angle of the knee.

Moreover, following distal resection the femur is shaped with the aid of a cutting block. To ensure correct shaping of the femur, the cutting block must be correctly positioned and sized. In particular, the cutting block must be correctly positioned with respect to the anterior-posterior direction and must be correctly rotated about an axis perpendicular to the distal resection plane such that the block's rotation corresponds to the correct Internal/External (I/E) rotation of the femur relative to the tibia. The I/E rotation may be set in a number of ways. One way of setting I/E rotation is by referencing the angle formed between the cutting block's medial-lateral axis as projected onto the distal resection plane and the knee's posterior condylar axis as projected onto the distal resection plane. In a typical case, the angle formed between the cutting block's medial-lateral axis as projected onto the distal resection plane and the knee's posterior condylar axis as projected onto the distal resection plane is set to approximately 3 degrees and matches the angle formed between the epicondylar axis as projected onto the distal resection plane and the posterior condylar axis as projected onto the distal resection plane.

A typical cutting block includes two or more fixation pegs, or “pins” that are used for positioning the block on the distal resection plane and securing the block to the plane. In practice, the block to be used is known and thus the positions of the pins within the block are known. Therefore, one can set the block's position in space by setting the pins' position in space. Accordingly, to position the block on the distal plane the appropriate pin positions are determined, pinholes are drilled at the determined positions, the pins in the block are lined up with the pinholes, and the pins are inserted into the pinholes to secure the block to the femur.

In many cases, the appropriate cutting block and the correct pinhole positions are determined using an instrument referred to as an “Anterior-Posterior Sizer” (or “AP Sizer”). The Sizer is designed to determine the appropriate cutting block and correct pinhole positions based on the type and size of femoral component that will be implanted. For example, the implant could be from the line of implants associated with the Stryker® Triathlon® Knee System which includes femoral implants of sizes 1-8. In such context, the AP Sizer will determine the size of Triathlon® implant that is needed and will indicate where the pinholes should be located for a cutting block corresponding to the Triathlon® implant of the determined size.

There are many different types of “sizing methodologies” employed for determining the correct implant size and hole position. For example, implant size and hole position can be determined through use of a “mechanical stylus,” a “blade runner,” or “drill sizing.” The type of sizing used in a procedure is often left to the discretion of the practitioner, with most practitioners having a preference for one method over the others. However, each of the prior methodologies requires the practitioner to estimate the correct implant size and hole position through direct visual inspection. Accordingly, the precision of the prior methodologies is limited by the error inherent in such direct visual inspection.

### SUMMARY OF THE INVENTION

In the interest of providing a sizing methodology of improved precision, the present invention was conceived. The invention provides an apparatus, method and system for sizing a distal portion of a femur during knee arthroplasty.

### DESCRIPTION OF THE DRAWINGS

The following detailed description, given by way of example, but not intended to limit the invention solely to the specific embodiments described, may best be understood in conjunction with the accompanying drawings wherein like reference numerals denote like elements and parts, in which:

FIG. 1A is a frontal view of the skeletal structure of a lower left hand portion of the human body.

FIG. 1B is a profile view of the portion shown in FIG. 1A.

FIG. 2 is a frontal view of the skeletal portion of a left knee joint in flexion.

FIG. 3 is perspective view of the knee joint of FIG. 2 as resected in preparation for implantation of articular bearing components of a resurfacing-type knee prostheses.

FIG. 4A is a perspective view of the knee joint of FIG. 3 in flexion with attached articular bearing components.

FIG. 4B is a cross-sectional profile view of the knee joint of FIG. 4A in extension.

FIG. 5 is a perspective view of a main unit and stylus of a femoral sizer in accordance with an embodiment of the invention.

FIG. 6 is a perspective view of the main unit of FIG. 5 attached to a distal portion of a femur.

FIG. 7 shows how the stylus of FIG. 5 interfaces with a tracker.

FIG. 8 is a perspective view of an assembled sizer attached to a distal portion of a femur.

FIG. 9 is a plan view showing how the assembled sizer of FIG. 8 is used in conjunction with a sensor array, computer, and display to size a distal portion of a patient's left femur.

FIG. 10 shows an example of an image displayed on the display of FIG. 9 during a process of femoral sizing.

FIG. 11 shows an example of an image displayed on the display of FIG. 9 during a process of setting I/E rotation.

### DETAILED DESCRIPTION OF PREFERRED EMBODIMENTS

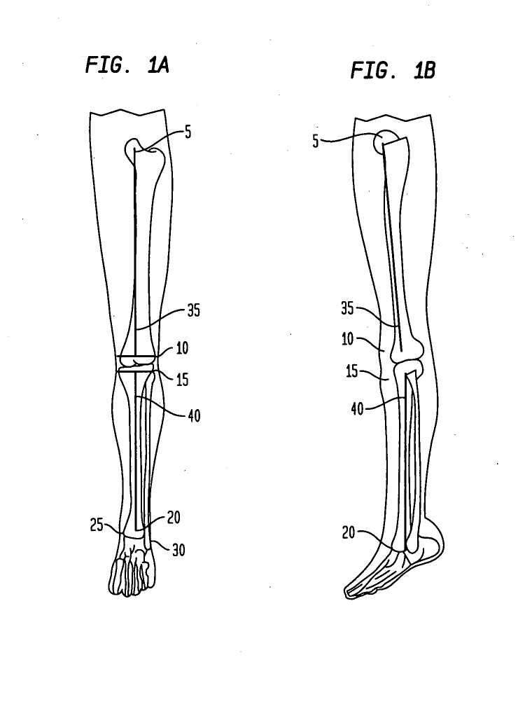

Prior to describing preferred embodiments of the invention in detail, an overview is provided. The overview concerns a knee arthroplasty procedure to which the invention is suited. The overview is provided with references to FIGS. 1A-4B.

Referring to FIG. 1A, there is shown a frontal view of the skeletal structure of a lower left hand portion of the human body. Several anatomical “landmarks” are defined. The landmarks include a center of the femoral head 5, a knee center 10, a tibia center 15, an ankle center 20, a medial malleolus 25, and a lateral malleolus 30. Further, a femoral axis 35 and a tibial axis 40 are defined. The femoral axis is defined by a line passing through the center of the femoral head and the center of the knee. The tibial axis is defined by a line passing through the tibia center and the ankle center.

FIG. 1B is a profile view of the portion shown in FIG. 1A.

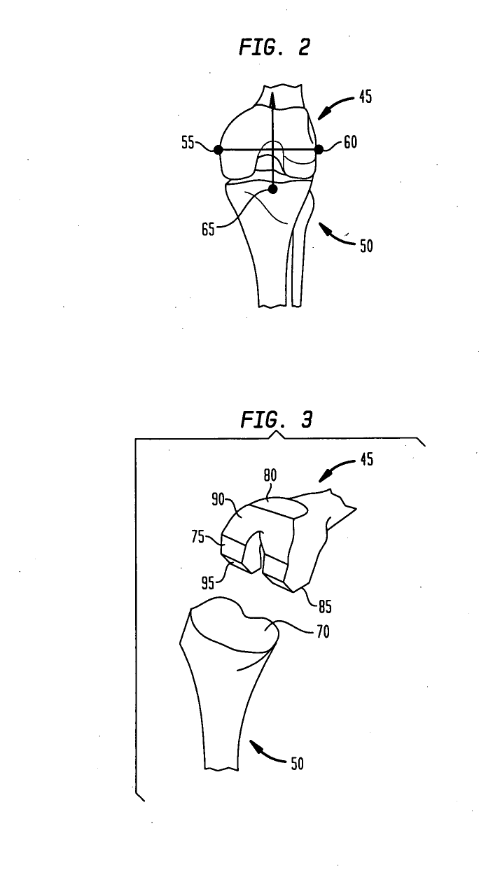

FIG. 2 is a frontal view of the skeletal portion of a left knee joint in flexion. As can be seen from FIG. 2, the joint is formed where a distal femur portion 45 meets a proximal tibia portion 50. Anatomical landmarks shown in FIG. 2 include a medial epicondyle 55, a lateral epicondyle 60, and an anterior-posterior axis (or “Whiteside Line”) 65. In order to implant articular bearing components of a resurfacing-type knee prostheses into the joint of FIG. 2, both the distal femur portion and the proximal tibia portion must be resected.

FIG. 3 is perspective view of the knee joint of FIG. 2 as resected in preparation for implantation of articular bearing components of a resurfacing-type knee prostheses. As can be seen from FIG. 3, the tibia has been resected along a single plane 70, the proximal tibial resection plane. The femur has been resected along five resection planes, a distal femoral resection plane 75, an anterior femoral resection plane 80, a posterior femoral resection plane 85, a distal-anterior femoral resection plane 90, and a distal-posterior femoral resection plane 95. The tibial and femoral resection planes are oriented so as to mate with the tibial and femoral bearing components.

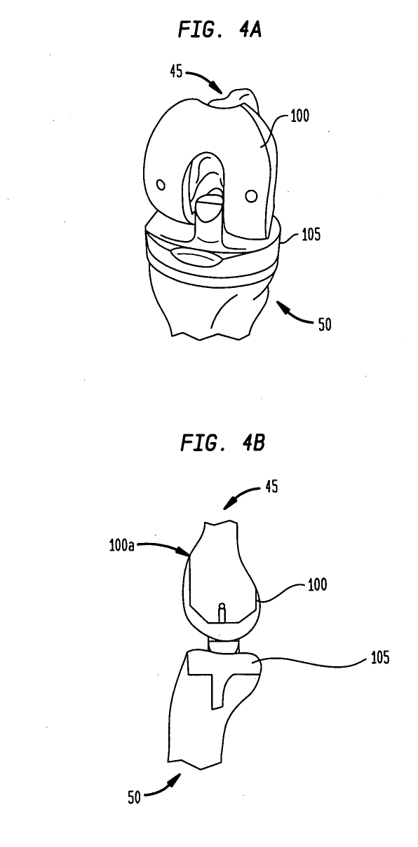

FIG. 4A is a perspective view of the knee joint of FIG. 3 in flexion with attached articular bearing components. As can be seen from FIG. 4A, a femoral bearing component (or “femoral implant”) 100 is mated with the distal femur portion 45, and a tibial bearing component (or “tibial implant”) 105 is mated with the proximal tibia portion 50.

FIG. 4B is a cross-sectional profile view of the knee joint of FIG. 4A in extension. From FIG. 4B it can be seen how the resection planes shown in FIG. 3 mate with surfaces of the femoral and tibial implants. In particular, a point 100a is noted. The point 100a is the “run-out point.” It is the most superior point of contact between the femoral implant and the femur, and it is critical to correct sizing and positioning of the femoral implant.

Having provided an overview of a knee arthroplasty procedure to which the invention is suited, a detailed description of preferred embodiments of the invention will now be provided.

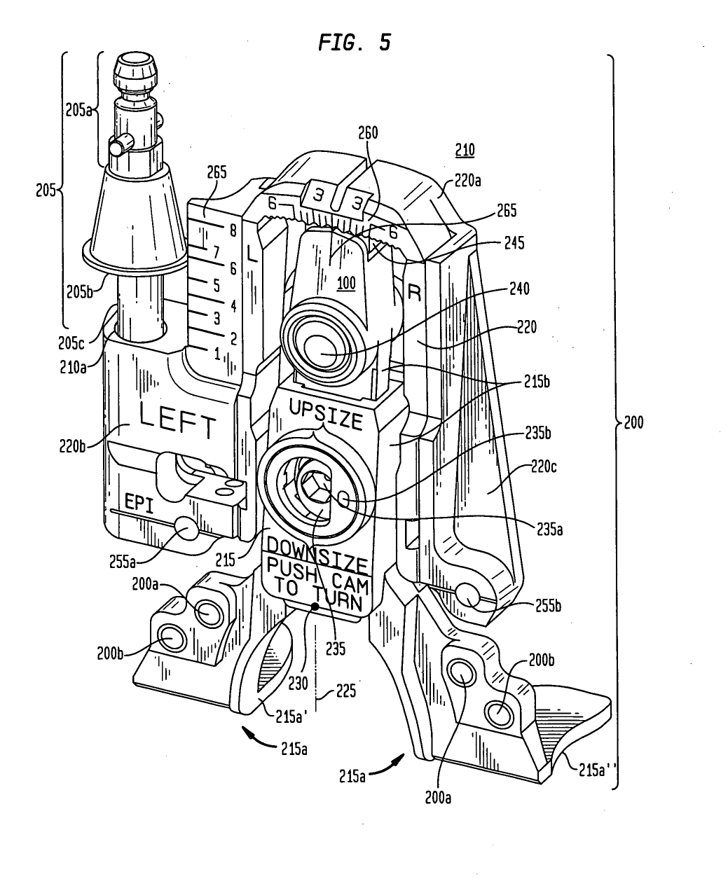

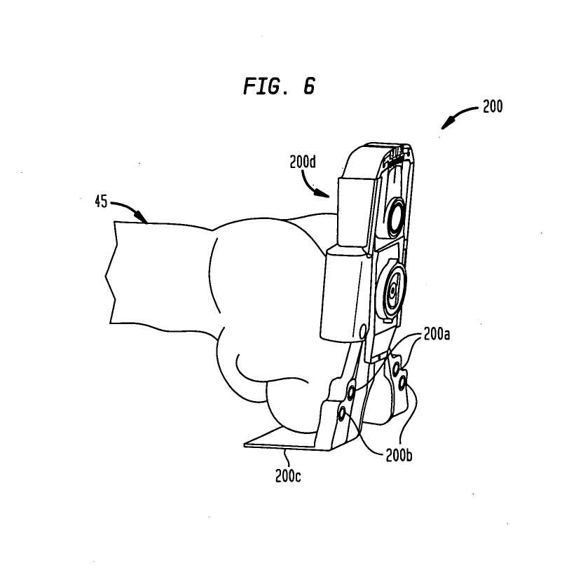

FIG. 5 is a perspective view of a main unit 200 and stylus 205 of a sizer assembly 210 in accordance with an embodiment of the invention. As shown in the figure, the stylus includes a head 205a to which a navigation tracker can be attached, a lip 205b which is used with a scale 265 on the main unit to obtain size readings, and a shaft 205c for attaching the stylus to the main unit. The longitudinal axis of the shaft is perpendicular to the plane defined by the lip. The shaft is accommodated in the main unit by positioning the shaft in a main unit through-hole 210a. Preferably, through-hole 210a accommodates the shaft such that the stylus is free to rotate about the longitudinal axis of the shaft and is free to translate longitudinally such that the stylus may move in a direction along the hole's longitudinal axis.

FIG. 6 shows how the main unit is attached to the distal femur portion for purposes of femoral sizing. The unit is attached to the distal femur after distal resection of the femur but before any other resections of the femur. For purposes of clarity, the stylus is not shown in FIG. 6.

As can be seen from FIG. 6, the main unit is attached to the distal femur portion by way of pinholes 200a and/or 200b, through which pins (not shown) are passed to secure the unit to the femur. Preferably, only one set of holes, either 200a or 200b is used to secure the unit, the choice being made according to the size of the femur to which the unit is attached. As can be further seen from FIG. 6, the unit includes a base portion 200c that contacts a posterior portion of the distal femur and a proximal face 200d that contacts the distal resection plane.

The main unit 200, best seen in FIG. 5, is made up a central part 215 and an outer part 220. The central part includes a base 215a and a trunk 215b. The outer part includes a top 220a and sides 220b and 220c. For purposes of this description, the portions of the central and outer parts that face away from the distal portion of the femur when the unit is attached to the femur will be referred to respectively as the “central part distal face” and “outer part distal face;” and the portions of the central and outer parts that face toward the distal portion of the femur when the unit is attached to the femur will be referred to respectively as the “central part proximal face” and “outer part proximal face.”

The outer part of the main unit and the central part of the main unit can be moved relative to one another. More specifically, the outer part can be both translated relative to the central part and rotated relative to the central part. The outer part can be translated relative to the central part in a direction parallel to the central part's longitudinal axis (as depicted by line 225). The outer part can be rotated relative to the central part about an axis perpendicular to the central part distal face, such as an axis perpendicular to the central part distal face and passing through point 230.

Movement of the outer part of the main unit relative to the central part of the main unit is controlled by two independently operated mechanisms. Translational movement of the outer part relative to the central part is controlled by a rotating element 235, and rotational movement of the outer part relative to the central part is controlled by a push-button 240.

To translate the outer part of the main unit relative to the central part, one inserts a suitably shaped instrument into a matching recess 235a in the rotation element, presses the element down toward the central part proximal face to unlock the element, and then rotates the instrument to rotate the element. A mechanical link causes the outer part to translate relative to the central part when the element is rotated. Preferably, the translational movement is infinitely variable within a predetermined range. Further, the rotating element preferably includes a detent 235b that mates with a protrusion on the central part of the main unit when the translation position is in the middle of the predetermined range, such that a positive confirmation of the middle position is provided.

To rotate the outer part of the main unit relative to the central part, one presses push-button 240 down toward the central part proximal face. The button is linked to a restraining element 245 having a multiple of teeth that mesh with teeth on the top of the outer part. When the button is pushed, the restraining element moves away from the top of the outer part (i.e. in a direction toward the base of the central part), and thereby the teeth of the restraining element are decoupled from the teeth on the top of the outer part. Once the restraining element and outer part are decoupled, the outer part is free to rotate about axis 230. After the outer part has been rotated to the desired position, the button is allowed to return to its original position, causing the restraining element to once again mesh with the top of the outer part, thereby locking the outer part in the desired position. The meshed teeth arrangement of the restraining element and the top of the outer part preferably provide rotation in 1 degree increments. However, it should be noted that the teeth can be arranged such that the increments are other than 1 degree. Moreover, the teeth can be eliminated so as to provide an infinitely variable adjustment.

In one embodiment, the main unit and stylus are used in conjunction with a tracker to perform femoral sizing. Accordingly, a femoral sizer according to one embodiment is formed of a main unit, stylus and tracker.

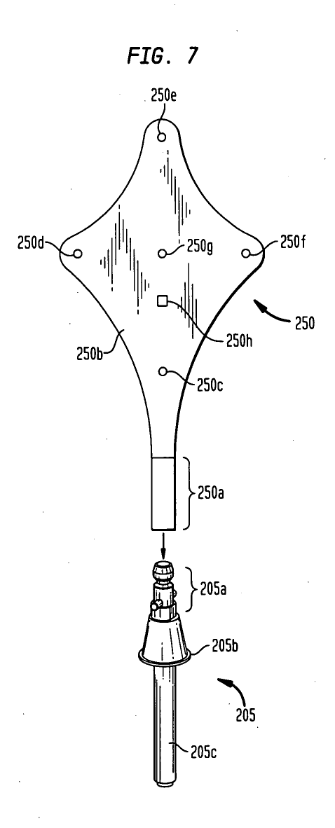

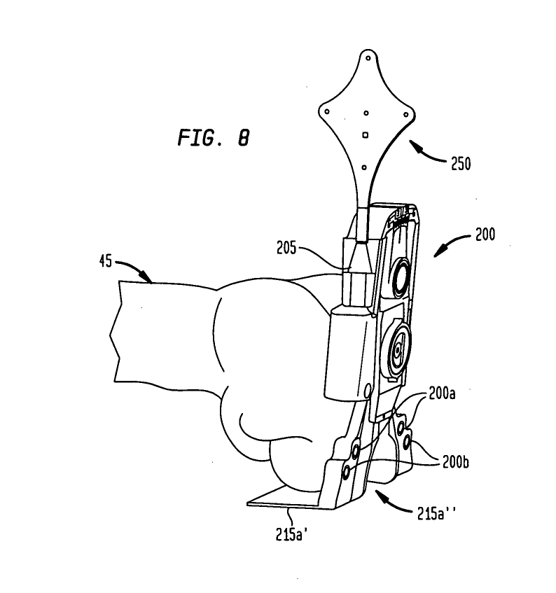

FIG. 7 is shows how the stylus 205 of FIG. 5 interfaces with a tracker 250. As can be seen from FIG. 7, the head of the stylus 205a mates with a receptacle 250a on the tracker. The longitudinal axis of the stylus shaft is perpendicular to the plane defined by lip 205b. The receptacle is part of a tracker body 250b, which also includes five transmitters 250c-250g. The transmitters are operable to signal a sensor array and/or computer that can determine the position and orientation of the tracker based on signals received from the transmitters. In a preferred embodiment, the transmitters are LEDs and transmission from transmitters is initiated through depression of an activation button 250h. In an alternative embodiment, reflectors are used in lieu of transmitters 250c-250g and the sensor array includes a transmitter for transmitting one or more signals that are reflected back to the array via the reflectors. The position and orientation of the tracker are determined according to the reflections received by the array.

The main unit 200, stylus 205 and tracker 250 make up a femoral sizer in accordance with one embodiment of the invention. The sizer is used to determine correct implant size and cutting block pin position by referencing the posterior condyles of the distal femur. FIG. 8 is a perspective view of such a sizer attached to the distal portion of a femur.

Referring to FIG. 8 in conjunction with FIG. 5, it can be seen that the sizer is aligned with the posterior condylar axis through two skids 215a′ and 215a′ located on the base of the main unit. The skids are positioned to contact the posterior condyles while the sizer is centered, or approximately centered, on the femur with respect to the medial-lateral direction.

Once the sizer is properly positioned, pins can be passed through either pair of pinholes 200a or 200b, or through both pairs of pinholes 200a and 200b, to secure the sizer to the femur. In a preferred embodiment, the main unit of the sizer is attached to the distal resection plane, the stylus is attached to the tracker, and then the stylus with attached tracker is attached to the main unit. Accordingly, it is not necessary that the stylus and tracker be attached to the main unit during attachment of the main unit to the femur.

In an alternative embodiment, the main unit 200, stylus 205, and tracker 250 are attached to each other to form a complete assembly, and then the complete assembly is attached to the femur via the pinholes.

In another embodiment, the stylus and tracker are parts of a single integrated unit. Such embodiment may be employed by attaching the integrated unit to the main unit to form a complete assembly and then attaching the complete assembly to the femur. Alternatively, such embodiment may be employed by attaching the main unit to the femur and then attaching the integrated unit to the main unit.

In still another preferred embodiment, the main unit and stylus are attached to each other, the main unit with stylus is attached to the femur, and then the tracker is attached to the stylus.

In yet another preferred embodiment, the stylus is attached to the main unit in a permanent fashion. That is, the stylus and main unit are permanently attached to each other such that they form a permanent sub-assembly. In such an embodiment, the preferred method of use is to attach the sub-assembly to the femur and then attach the tracker to the main unit.

It should be noted that the words “attach,” “attached,” and “attaching” as used in this description are not limited to the case of attaching in the permanent sense, but rather, contemplate both the case of attaching in the permanent sense and the case of attaching in the removable sense.

In any event, once the main unit of the sizer is correctly positioned on the femur, the internal-external rotation of the implant is set by setting the internal-external rotation of the main unit. In this regard, it is important to note that the main unit includes two drill guide holes 255a and 255b (see FIG. 5), which relate to a cutting block type which, in turn relates to a type of implant. Upon final positioning of the main unit, holes are drilled in the femur at positions determined by the guide holes. Thus, the main unit position determines the guide hole positions, which determines the cutting block position which, in turn, determines the implant position. Therefore, by setting the internal-external rotation of the main unit, the internal-external rotation of the implant is being set.

To set the internal-external rotation of the main unit, the outer part of the unit is rotated relative to the central part of the unit. Since the central part is fixed relative to the posterior condylar axis, and both the central and outer parts are fixed relative to the distal resection plane, rotation of the outer part relative to the center part has the effect of changing the inclination between the posterior condylar axis as projected onto the distal resection plane and an imaginary line connecting the drill guide holes as projected onto the distal resection plane. The change in magnitude of such inclination is equal to the magnitude of the internal-external rotation.

The outer part of the main unit is rotated by depressing push-button 240, moving the outer part to the desired position, and then releasing the push button to lock the outer part in place. The degree of internal-external rotation is read from a scale 260 located at the top of the outer part. The scale is referenced to a mark 265 on the central part.

After the internal-external rotation of the implant is set, the sizer can be used to size the femur. That is, the sizer can be used to determine the appropriate size implant needed for the subject femur.

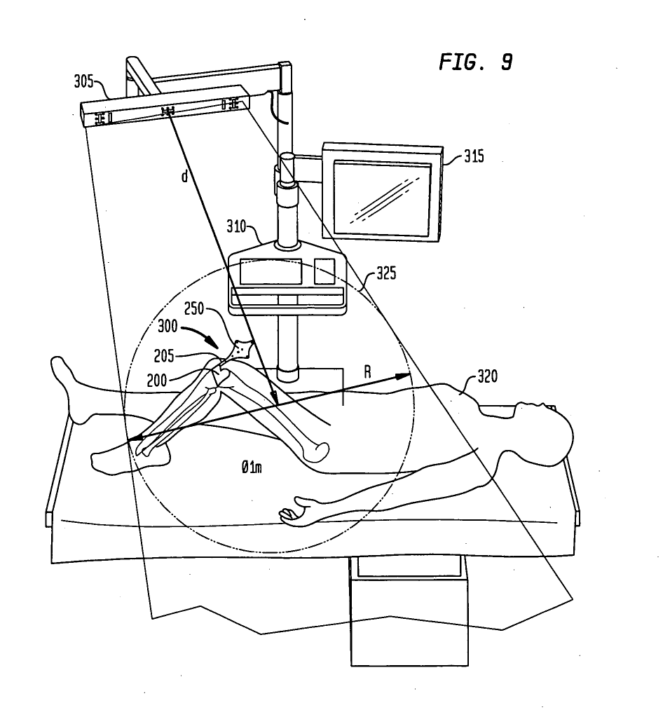

FIG. 9 is a plan view showing how a sizer 300 like that shown in FIG. 8 is used in conjunction with a sensor array 305, a computer 310, and a display 315 to size a left distal femur portion of a patient 320. As can be seen from FIG. 9, the sizer is attached to the patient's left femur and includes main unit 200, stylus 205, and tracker 250. The patient is positioned such that the tracker is in a field of view 325 of the sensor array. The field of view is generally defined by a sphere of radius “R” having its center a distance “d” from the center of the sensor array and being located on a line extending from the center of the array and being perpendicular to the frontal plane of the array.

The tracker signals the sensor array. In the preferred embodiment, the tracker includes light emitting diodes (LEDs) such as LEDs 250c-250g of FIG. 7 and the tracker signals the sensor array via transmissions from the LEDs to the sensor array. In any case, the signals received by the sensor array are converted to computer signals, and the computer signals are passed to the computer and used by the computer to generate a virtual three-dimensional Cartesian coordinate system (x-axis, y-axis, and z-axis) that is fixed in space relative to the tracker. Any movement of the tracker results in corresponding movement of the three-dimensional coordinate system. Further, the three dimensional coordinate system is established so that one of the planes defining the system (e.g. the x-y plane) is parallel to the plane defined by stylus lip 205b. Thus, the coordinate axis perpendicular to the lip plane (e.g. the z axis) is parallel to the longitudinal axis of the stylus shaft 205c. Moreover, through-hole 210a (best seen in FIG. 5) is inclined relative to the longitudinal axis of the main unit such that when the main unit is attached to the distal resection plane, the stylus is inserted in the main unit, and the sizer is set to the correct I/E rotation, the lip plane is parallel to the plane of the anterior resection (i.e. the “anterior resection plane”).

In a preferred embodiment, the lip plane and anterior resection plane are co-planar when the sizer is set to the correct I/E rotation. In alternative embodiments, the lip plane and anterior resection plane are offset relative to one another when the sizer is set to the correct I/E rotation, the anterior resection plane being either above or below the lip plane. This description contemplates both embodiments in which the lip plane and anterior resection plane are co-planar when the sizer is set to the correct I/E rotation and embodiments in which the lip plane and anterior resection plane are offset relative to one another when the sizer is set to the correct I/E rotation.

Referring back to the sizing procedure, a second tracker is used to map anatomical landmarks of the patient. The second tracker is preferably a hand-held tracker and preferably signals the sensor array in the same manner that tracker 250 signals the array. The landmarks that are mapped are selected landmarks relevant to correct sizing of the patient's femur. Such landmarks may include, for example, the landmarks shown in FIGS. 1A, 1B and 2. In any case, signals generated by the second tracker during mapping of the landmarks are received by the sensor array which converts the signals to computer signals and transmits such computer signals to the computer. The computer then computes the position of the landmarks based on the computer signals.

Once the sizer has been attached to the femur and the landmark positions have been established, the stylus can be translated within through-hole 210a of the main unit (see FIG. 5) to size the femur. As the stylus is translated within through-hole 210a, the computer compares the position of the anterior resection plane (lip plane) defined by tracker 250 to the position of the landmarks. When the stylus is positioned such that the anterior resection plane intersects the run-out point desired by the practitioner (“the desired run-out point”), the sizing is complete. The size reading is taken by observing the position of lip 205b relative to scale 265 (see FIG. 5). For example, if the lip is pointed toward the number “3” of the scale, then the femur/implant size is “3.”

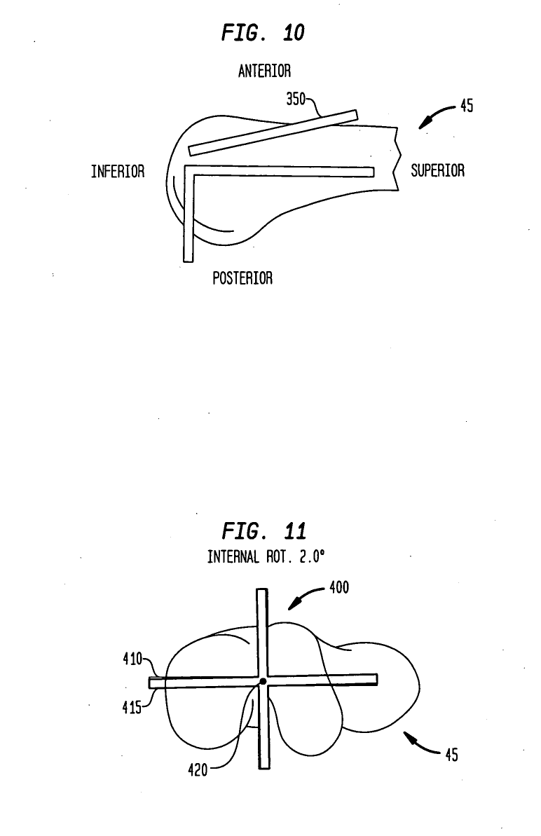

In order to determine when the stylus is positioned such that the anterior resection plane intersects the desired run-out point, an image of the position of the anterior resection plane relative to the femur is shown on display 315. FIG. 10 shows an example of such an image.

The image displayed in FIG. 10 is that of the distal femur portion and anterior resection plane as viewed from a direction parallel to the anterior resection plane. Accordingly, as can be seen from FIG. 10, the anterior resection plane is represented by a line 350. If during a sizing procedure the practitioner were to move the stylus in an anterior direction, such movement would be reflected on the display as upward movement of line 350. Likewise, if the practitioner were to move the stylus in a posterior direction, such movement would be reflected on the display as downward movement of line 350. In this manner, the practitioner can move the stylus while observing the display. When the stylus is positioned such that the anterior resection plane runs-out of the femur at the desired run-out point, then the stylus is correctly positioned and the practitioner can read the femoral implant size by observing the position of stylus lip 205b relative to scale 265.

If the stylus lip is between the markings indicated on scale 265 when the display indicates that the anterior resection plane runs-out of the femur at the desired run-out point, then the femur is “between sizes.” In such an event, the femur may be said to be of the size that most closely approximates the actual stylus position. However, if sizing is conducted in this manner, the resulting run-out point will not be the desired run-out, or in other words, the implant will not be ideally positioned. In common terminology, a mismatch in size measurements is an indication that the implant may “notch” or “overhang.”

The sizer provides a mechanism for adjusting position of the implant to avoid “notching” and “overhanging.” More particularly, when achieving the desired run-out point results in a stylus lip position that is between sizes on scale 265, the practitioner moves the stylus such that the lip moves to the position adjacent the size that most closely corresponds to the desired run-out point. In this condition, the practitioner can observe on the display the degree to which the implant will “notch” or “overhang.” If line 350 intersects the anterior femur at a point inferior to the desired run-out point, the distance from the intersection point to the desired run-out point indicates the “overhang” magnitude. If line 350 intersects the anterior femur at a point superior to the desired run-out point, the distance from the intersection point to the desired run-out point indicates the “notch” magnitude.

In any event, when a “notch” or “overhang” is indicated, a correction can be made by shifting the implant. That is, for a given size implant, the implant can be shifted such that it properly mates with the desired run-out point. This is done using rotating element 235 to translate the outer part of the main unit relative to the central part. For example, in an “overhanging” situation the rotating element is used to move the outer part in a generally posterior direction which, in turn, moves the drill guide holes 255a and 255b in the generally posterior direction. Since the hole position corresponds to the cutting block and implant position, movement of the drill guide holes in a generally posterior direction results in corresponding movement of the implant in the generally posterior direction. In this manner, the run-out point for a given size implant can be adjusted to correct for an “overhanging” situation.

In a similar manner, a shift of implant position can be made to correct a “notching” situation. To correct for a “notching” situation, the rotating element may be used to move the outer part in a generally anterior direction which, in turn, moves the implant in the generally anterior direction.

It should be noted that the stylus, tracker and display may also be used to aid in setting the internal-external rotation of the sizer. To set the internal-external rotation of the sizer with the aid of the stylus, tracker and display, a practitioner attaches the sizer to the patient's femur as shown in FIG. 9, depresses button 240 (see FIG. 5), and rotates the outer part of the main unit relative to the central part of the main unit. While rotating the outer part, the practitioner observes on display 315 an image depicting the I/E rotation. The image provides the practitioner with an indication of I/E rotation. Thus, the practitioner can use the image to confirm I/E settings, or may base the I/E settings solely on the image. An example of such an image is shown in FIG. 11.

The image of FIG. 11 includes both a numerical indication of I/E rotation 400 and a graphical indication of I/E rotation. In the illustrated case, the I/E rotation is 2.0 degrees internal rotation.

Regarding the graphical indication of rotation, two cross-hairs 410 and 415 sharing a center 420 are presented as superimposed on an image of the distal femur portion. Cross-hair 410 represents the “neutral” position, or “no I/E rotation” position. Cross-hair 415 represents the current I/E rotation of the sizer as indicated by the position and orientation of the tracker relative to the mapped landmarks. Thus, if button 240 of the sizer is depressed, and the outer part of the main unit is rotated relative to the central part such rotation will be reflected by a change in rotation of cross-hair 415 about center 420. The resulting I/E rotation can be judged by observing the resulting relative rotation of cross-hairs 410 and 415.

As these and other variations and combinations of the features discussed above can be utilized without departing from the present invention as defined by the claims, the foregoing description of the preferred embodiments should be taken by way of illustration rather than by way of limitation of the invention as defined by the claims. For example, the foregoing description was provided in the context of left-knee arthroplasty. However, upon review of the description, one skilled in the art will readily appreciate how the invention is implemented in right-knee arthroplasty.

1. An apparatus for sizing a distal portion of a femur during knee arthroplasty, comprising:
a main unit that can be attached to the distal portion of the femur; and
a stylus operable to support a tracker and operable to be attached to the main unit such that the stylus can move relative to the main unit.

2. The apparatus as set forth in claim 1, wherein the stylus is permanently attached to the main unit.

3. The apparatus as set forth in claim 1, wherein the stylus is operable to be attached to the main unit such that the stylus can translate in at least one direction relative to the main unit.

4. The apparatus as set forth in claim 1, wherein the stylus comprises a shaft for attaching the stylus to the main unit such that the stylus can translate along the longitudinal axis of the shaft.

5. The apparatus as set forth in claim 1, wherein the stylus is operable to be attached to the main unit such that the stylus can rotate about at least one axis.

6. The apparatus as set forth in claim 1, wherein the stylus comprises a shaft for attaching the stylus to the main unit such that the stylus can rotate about the longitudinal axis of the shaft.

7. The apparatus as set forth in claim 1, further comprising a tracker.

8. The apparatus as set forth in claim 7, wherein the stylus and tracker are parts of an integrated unit.

9. The apparatus as set forth in claim 8, wherein the tracker is operable to transmit signals.

10. The apparatus as set forth in claim 9, wherein the tracker comprises at least one light emitting diode (LED), and wherein the at least one LED is operable to transmit optical signals to a sensor array.

11. A system for sizing a distal portion of a femur during knee arthroplasty, comprising:
a sensor array;
a computer; and
a femoral sizer, the femoral sizer comprising,
a main unit that can be attached to the distal portion of the femur;
a stylus operable to be attached to the main unit such that the stylus can move relative to the main unit; and
a tracker operable to be attached to the stylus and operable to transmit at least one signal to the sensor array;
wherein the sensor array is operable to convert the at least one signal received by the sensor array to at least one computer signal and transmit the at least one computer signal to the computer, and the computer is operable to use the at least one computer signal to generate an indication used in sizing the distal portion of the femur.

12. The system as set forth in claim 11, wherein the stylus is permanently attached to the main unit.

13. The system as set forth in claim 11, wherein the stylus is operable to be attached to the main unit such that the stylus can translate in at least one direction relative to the main unit.

14. The system as set forth in claim 11, wherein the stylus comprises a shaft for attaching the stylus to the main unit such that the stylus can translate along the longitudinal axis of the shaft.

15. The system as set forth in claim 11, wherein the stylus is operable to be attached to the main unit such that the stylus can rotate about at least one axis.

16. The system as set forth in claim 11, wherein the stylus comprises a shaft for attaching the stylus to the main unit such that the stylus can rotate about the longitudinal axis of the shaft.

17. The system as set forth in claim 11, wherein the tracker comprises at least one light emitting diode (LED), and wherein the at least one LED is operable to transmit optical signals to the sensor array.

18. The system as set forth in claim 11, further comprising a display for displaying the indication used in sizing the distal portion of the femur.

19. A method for sizing a distal portion of a patient's femur during knee arthroplasty, comprising the steps of:
positioning the patient such that a distal femur portion of the patient is in a field of view of a sensor array;
attaching a femoral sizer to the distal femur portion, the femoral sizer including a tracker that is operable to provide signals to the sensor array;
manipulating the sizer while observing a display that displays an image based on the signals provided by the tracker to the sensor array; and
determining the size of the distal portion of the femur by observing the sizer in response to an indication provided on the display.

20. The method as set forth in claim 19, wherein the step of attaching comprises the steps of assembling the femoral sizer and attaching the assembled sizer to the patient's femur.

21. The method as set forth in claim 19, wherein the sizer comprises a main unit, a stylus and the tracker.

22. The method as set forth in claim 19, further comprising the step of mapping at least one of the patient's anatomical landmarks.

23. The method as set forth in claim 19, further comprising the step of determining an Internal/External (I/E) rotation setting for the sizer.
